(19) United States
(12) Patent Application Publication
(10) Pub. No.: US 2007/0003600 A1
(43) Pub. Date: Jan. 4, 2007

(54) Methods for reducing c-reactive protein

(76) Inventors: Carolyn Moore, Houston, TX (US); Erich P. Lerchenfeld, Tulsa, OK (US); Donald E. Striegel, Clemont, FL (US); Ishwarlal Jialal, Sacramento, CA (US); Sridevi Devaraj, Sacramento, CA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/315,206

(22) Filed: Dec. 23, 2005

Related U.S. Application Data
(63) Continuation-in-part of application No. 10/458,692, filed on Jun. 11, 2003.
(63) Continuation-in-part of application No. 10/691,581, filed on Oct. 24, 2003.

(51) Int. Cl. A61K 36/752; A61K 31/56; A61K 47/00

(52) U.S. Cl. 424/439; 424/736; 514/169

(57) ABSTRACT

The present disclosure is directed to a method for reducing the level of c-reactive protein comprising administering to a subject in need thereof a c-reactive protein level reducing amount of at least one phytosterol.

This application is a continuation-in-part of and claims the benefit of U.S. application Ser. Nos. 10/458,692, filed Jun. 11, 2003, and Ser. No. 10/691,581, filed Oct. 24, 2003, the contents of which are incorporated herein by reference.

The present disclosure relates to methods for reducing the level of c-reactive protein comprising administering to a subject in need thereof, a c-reactive protein level reducing amount of at least one phytosterol.

Inflammation plays a critical role in atherosclerosis (cardiovascular disease) and evidence suggests inflammation is present throughout the developmental stages of atherosclerosis, i.e., fatty streak to acute coronary syndromes. See R. Ross, Atherosclerosis: An Inflammatory Disease, 340 N. Engl. J. Med. 115-26 (199); P. Libby, Inflammation in Atherosclerosis, 420 Nature 868-74 (2002). Given its prevalence, signals indicating inflammation can offer important clues for prevention, progression, and even, monitoring purposes. According to numerous studies, c-reactive protein (CRP) is an inflammatory marker present in the blood that may help assess cardiovascular disease.

CRP has long been used to monitor rheumatology, i.e., the activity of rheumatoid arthritis, and has recently been shown to be an independent marker for cardiovascular disease. See e.g., lshwarlal Jial et al., C-Reactive Protein: Risk Marker or Mediator in Atherothrombosis, 44 Hypertension 6-11 (2004). In fact, the American Heart Association and Centers for Disease Control and Prevention issued statements recommending CRP be used as a risk marker for cardiovascular disease with a Framingham risk score of between 10% and 20%. Based on their recommendations, CRP levels <1 mg/L are considered low risk, levels from 1 to 3 mg/L represents average risk, and levels>3 mg/L are considered high risk.

CRP is a member of the pentraxin family of proteins; pentraxins are known to form pentameric complexes and characteristically can bind numerous ligands. Shrive, A. K., C-Reactive Protein and SAP-like Pentraxin are Both Present in Limulus Polyphemus Haemolympa: Crystal Structure of Limulus SAP, 290 J. Mol. Bio. 997-1008 (1999). For example, CRP's cyclic pentameric structure includes five non-covalently associated protomers arranged around a central pore with a molecular weight around 118 000 Da. Thompson D. et al., The Physiological Structure of Human C-Reactive Protein and Its Complex with Phosphocholine, 7 Structure 169-77 (1999). CRP is nonglycosylated and is mapped to chromosome 1. Senthil Kumar et al., Effect of C-Reactive Protein on Vascular Cells: Evidence for a Proinflammatory, Proatherogenic Role, 14 Current Opinion in Nephrology & Hypertension 33, 34 (2005).

According to historic research, CRP production resided in the liver and was driven by interleukin (IL)-6 with synergistic effects of IL-1 in hepatocytes. I. Kushner et al., Control of the Acute Phase Response: C-Reactive Protein Synthesis by Isolated Perfused Rabbit Livers, 96 J. Lab. Clinical Med. 1037-1045 (1980); I. Kushner et al., Control of the Acute Phase Response: Demonstration of C-Reactive Protein Synthesis and Secretion by Hepatocytes During Acute Inflammation in the Rabbit, 148 J. Ex. Med. 466-77 (1978). Based on recent studies, CRP production, however, may also be found in other tissues such as atherosclerotic lesions, alveolar macrophages, neuronal cells, tubular epithelial cells, and human aortic endothelial cells. K. Yasojima et al., Generation of C-Reactive Protein and Complement Components in Atherosclerotic Plaques, 158 Am. J. Pathol. 1039-51 (1989); S. Kobayashi, Interaction of Oxidative Stress and Inflammatory Response in Coronary Plaque Instability: Important Role of C-Reactive Protein, 23 Arterioscler Thromb. Vasc. Biol. 1398-1404 (2003); G. D. Reynolds and R. P. Vance, C-Reactive Protein Immunohistochemical Localization in Normal and Atherosclerotic Human Aortas, 111 Arch. Pathol. Lab. Med. 265-69 (1987); Q. Dong and J. R. Wright, Expression of C-Reactive Protein by Alveolar Macrophages, 156 J. Immunol. 4815-20 (1996); K. Yasojima et al., Human Neurons Generate C-Reactive Protein and Amyloid P: Upregulation in Alzheimer's Disease, 887 Brain Res. 80-89 (2000); W. J. Jabs et al., The Kidney as a Second Site of Human C-Reactive Protein Formation In Vivo, 33 Eur. J. Immunol. 152-61; and Senthil Kumar Venugopal et al., Macrophage Conditioned Medium Induces the Expression of C-Reactive Protein in Human Aortic Endothelial Cells, 166 American J. Pathology 1265-71 (2005). Data also suggest that CRP production may be triggered by lipid peroxidation, infections and viral agents such as cytomegalovirus, herpes simplex virus, Chlamydia pneumoniae, and Helicobacter pylori.

Given CRP production triggers, evidence suggests that CRP acts on monocyte/macrophages, endothelia cells, and smooth muscle cells. In these cells, CRP stimulates the secretion of a wide variety of proinflammatory molecules. These proinflammatory molecules have been shown to be present through the various stages of atherosclerosis. Isthwarlal Jialal et al., C-Reactive Protein: Risk Marker or Mediator in Atherothrombosis? 44 Hypertension 6-11 (2004). In fact, data suggests CRP may not only be a risk marker for cardiovascular disease but also may play a role in atherogenesis. Id. According to data, endothelial vasoreactivity shows an inverse relationship with CRP levels. S. Fichtlscherer et al., Elevated C-Reactive Protein Levels and Impaired Endothelial Vasoreactivity in Patients with Coronary Artery Disease, 102 Circulation 1000-1006 (2000); S. J. Cleland et al., Endothelial Dysfunction as a Possible Link Between C-Reactive Protein Levels and Cardiovascular Disease, 98 Clinical Science (London) 531-35 (2000); F. Tomai et al., Unstable Angina and Elevated C-Reactive Protein Levels Predict Enhanced Vasoreactivity of the Culprit Lesion, 104 Circulation 1471-76 (2001).

At this time, treatments such as for weight loss in obese individuals and certain medications, i.e., 3-hydroxy-3-methylglutaryl coenzyme A reductase inhibitors (HMG-CoA reductase inhibitors), e.g., statins, peroxisome proliferators-activated receptor-α agonists (fibrates), peroxisomes proliferators-activated receptor-α agonists (glitazones), aspirin, and high doses of RRR-α tocopherol, may be used to regulate the level of high sensitivity (hs)-CRP. Each of these treatments, however, has potential limitations and disadvantages.

For example, patients on statin therapy, as with any type of pharmaceutical therapy, inherently run the risk of side effects and/or adverse events from the respective drug, i.e., safety concerns, and they have the potential to cause problems in some people more so than others, e.g., the elderly. In addition, statins are often taken for long periods of time and their potential long term effects may not yet be apparent. With statins, there is an enhanced concern regarding the onset of muscle problems, e.g., rhabdomyolysis. Rhabdomyolysis results in a severe breakdown of muscle tissue that may be toxic to the kidneys, which can ultimately lead to kidney failure and death. Recently, the statin cerivastatin was pulled from the market based on increased incidences of rhabdomyolysis associated with high doses, as well as from combination doses with gemfibrozil. Other common side effects of statin therapy include cognitive problems, gastrointestinal and neurological effects and immune effects. Based on at least these concerns, the potential side effects and safety considerations may outweigh the benefits of the therapy, at least in some instances.

Although most of these treatments are seeing appreciable advancements, it remains desirable to reduce CRP levels, while at the same time decreasing at least one side effect associated with the various traditional therapies. While investigating methods of preventing and/or reducing atherosclerosis, the present inventors discovered that plant sterols and/or stanols, i.e., phytosterols, are useful in reducing c-reactive protein.

Plant sterols occur naturally in vegetable oils. Plant stanols also occur naturally, but are hydrogenation compounds of a corresponding plant sterol. As early as the 1950's, the scientific literature reported that plant sterols have some effect in reducing atherosclerotic events in mammals, i.e., reduction in blood serum cholesterol in man, and the reduction of serum cholesterol in young men with atherosclerotic heart disease. Pollak, O. J., Successful Prevention of Experimental Hypercholesterolemia and Cholesterol Atherosclerosis in the Rabbit, 7 Circulation 696-701 (1953); Farquhar et al., The Effect of Beta Sitosterol on the Serum Lipids of Young Men with Arthrosclerosis Disease, 14 Circulation 77-82 (1956). It is known that plant sterols and stanols exhibit cholesterol lowering effects by preventing the absorption of cholesterol in the small intestines. See, e.g., Mattson, FH, Grundy, SM, & Crouse, JE, Optimizing the effect of plant sterols on cholesterol absorption in man, 35 Am. J. Clin. Nut. 697-700 (1982). Other scientific literature establishes that plant sterols and stanols do, in fact, lower the level of serum cholesterol in humans, however, because of poor solubility in water, it has been difficult to prepare products suitable for human and veterinary consumption that contained these plant sterols or stanols.

Generally, plant sterols and stanols have been employed in margarines and other so-called spreads or similar food products because of their hydrophobic properties. U.S. Pat. Nos. 3,881,005 and 4,195,084, both assigned to Eli Lilly, describe grinding or milling plant sterols in order to enhance their solubility. Eli Lilly at one time marketed a sterol preparation from tall oil and later from soybean oil under the trademark Cytellin that lowered serum cholesterol by about 9%. Kuccodkar et al., Effects of Plant Sterols on Cholesterol Metabolism in Man, 23 Atherosclerosis 239-48 (1976). The product, however, never received widespread acceptance.

Vulfson et al., WO 00/41491 discloses hydrophobic compounds such as plant sterols and lycopenes as supplements to food products and beverages such as oleomargarine products, drinks, soups, sauces, dips, salad dressings, mayonnaise, confectionary products, breads, cakes, biscuits, breakfast cereals, and yogurt type products. Vulson et al., in combining the plant sterol or lycopene with the product, theorized that the food product which has both hydroxyl and carboxyl groups interacts with the surface of the sterol or lycopene.

U.S. Pat. No. 6,572,876 is also directed to a composition containing plant sterols, soy protein, and isoflavins and combinations thereof, which are useful for lowering LDL-cholesterol and total cholesterol blood concentrations and for preventing or minimizing development of atherosclerosis. Although plant sterols and/or stanols were known to impact serum cholesterol levels, plant sterols and/or stanols were not known to be effective on CRP levels.

In fact, in Steven E. Nissen et al., Statin Therapy, LDL Cholesterol, C-Reactive Protein and Coronary Artery Disease, 352 N. Engl. J. Med. 29-38 (2005), data suggest that changes in CRP levels and LDL-cholesterol levels are independent predictors of plaque regression with statin therapy. Likewise in Paul M. Ridker et al., C-Reactive Protein Levels and Outcomes After Statin Therapy, 352 N. Engl. J. Med. 20, 26 (2005), data support the assertion that “CRP levels independently predict the risk of first coronary events at all levels of LDL-cholesterol and across a full spectrum of Framingham risk categories and that CRP levels have prognostic value in patients with active coronary syndromes.” Accordingly, an association between LDL-cholesterol and CRP is missing and instead, CRP is viewed as an independent marker for cardiovascular disease.

The present disclosure accordingly proposes methods for reducing levels of c-reactive protein comprising administering to a subject in need thereof a c-reactive protein level reducing amount of at least one phytosterol.

Accordingly, the present disclosure relates to, among other things, a method for reducing the level of c-reactive protein comprising administering to a subject in need thereof a c-reactive protein level reducing amount of at least one phytosterol.

In a further embodiment, the present disclosure is directed to a method for treating or preventing vascular inflammation comprising administering to a subject in need thereof a c-reactive protein level reducing amount of at least one phytosterol.

Another embodiment of the present disclosure is directed to a method for reducing the levels of c-reactive protein comprising administering to a subject in need thereof a beverage comprising a substantially stable dispersion of at least one phytosterol in a c-reactive protein level reducing amount and an aqueous material wherein the at least one phytosterol is chosen from plant sterols and plant stanols, wherein in order to avoid a powdery taste in the substantially stable dispersion, the particle size of the at least one phytosterol is from 0.1 micron to about 30 microns and a majority of the at least one phytosterol particles are within a range from about 0.2 microns to about 10 microns and follow a bell curve distribution.

Additional advantages of the present disclosure will be set forth in part in the description which follows, and in part will be obvious from the description, or may be learned by practice of the present disclosure. The advantages of the present disclosure will be realized and attained by means of the elements and combinations particularly pointed out in the appended claims.

It is to be understood that both the foregoing general description and the following detailed description are exemplary and explanatory only and are not restrictive of the present disclosure, as claimed.

Accordingly, the present disclosure is directed to methods for reducing the level of c-reactive protein comprising administering to a subject in need thereof a c-reactive protein level reducing amount of at least one phytosterol. To administer the at least one phytosterol, the at least one phytosterol may be a component of a composition. For example, a composition may be, in a form chosen from a pharmaceutical and a consumable food product such as a solid or semi-solid food product, a nutraceutical, i.e., functional food, or a liquid product, e.g., a beverage.

In at least one embodiment, the composition is a nutritional substance, i.e., a consumable product and/or nutraceutical, which a subject may be able to consume on a daily basis. Mention may be made, for example, of nutritional beverages, soft drinks, fruit beverages and juices, electrolyte containing beverages, puddings, baked goods, non-baked goods, salad dressings, cereal products, condiments, confections, snack foods, dips and spreads, ice cream, frozen confections and novelties, dairy products such as yogurts, margarine-like spreads, and seasonings. In addition, fat free, reduced-fat and low calorie versions of these foods and beverages are also contemplated by the present disclosure.

As further example, the at least one phytosterol of the present disclosure may be incorporated into a pharmaceutical composition such as a tablet, an injection, or any other vehicle known to a skilled artisan to administer the at least one phytosterol. The pharmaceutical composition may also be formulated in such a manner known to those skilled in the art so that the composition exhibits a release profile chosen from immediate, modified, and delayed-release profiles.

In addition, administration may take the form of any other vehicle known to a skilled artisan conducive to facilitate a subject's ingestion of the at least one phytosterol.

Examples of suitable subjects that may be treated according to the methods of the present disclosure include mammals, such as humans, dogs, or other animals.

Available data identifies mechanisms in which plant sterols and stanols inhibit cholesterol, but the mechanism through which plant sterols and stanols modulate CRP levels is not understood. Without wishing to be bound by any particular theory, the present inventors believe that an ordinary intake level of phytosterols may have little influence on the level of CRP, but the intake of, for example, above an ordinary level and/or a particular form of phytosterols, may influence the level of CRP through normal bodily functions.

Preparation of a Composition

As used herein, the term “phytosterol” refers to plant sterols and plant stanols in their free and esterified forms with e.g., a fatty acid ester of sitosterol. The at least one phytosterol disclosed herein may be used in the free form. Plant sterols are naturally occurring compounds present in minor amounts in a number of food sources such as fruits, vegetables, nuts, seeds, cereals, legumes, and vegetable oils. Scientific literature describes at least 44 plant sterols, and the skilled artisan may choose any plant sterol and from those that are available when practicing the present disclosure. The present disclosure, also involves using some of the plant sterols employed in the art. For example, mention may be made of plant sterols including sitosterol, campesterol, stigmasterol, spinosterol, taraxasterol, brassicasterol, desmosterol, chalinosterol, poriferasterol, clionasterol, and ergosterol. The present disclosure also employs mixtures of plant sterols, such as two component, three component, and four component mixtures.

The source of these and other plant sterols may be from, for example, rice bran, corn bran, corn germ, wheat germ oil, corn oil, safflower oil, oat oil, olive oil, cotton seed oil, soybean oil, peanut oil, black tea, green tea, colocsia, kale, broccoli, sesame seeds, shea oils, grapeseed oil, rapeseed oil, linseed oil, canola oil, tall oil and other oils obtained from wood pulp. Table I below summaries the phytosterol content of some common vegetable fats provided in the article: Richard E. Ostlund et al., Effects of Trace Components of Dietary Fat on Cholesterol Metabolism: Phytosterols, Oxysterols, and Squalene, 60 Nutrition Reviews 349-59 (2002).

TABLE I
Phytosterol Content of Common Vegetable Fats

| Source | Phytosterol (mg/100 g) |
|---|---|
| Nutmeg | 0 |
| Coconut | 86 |
| Uchuba Butter | 95 |
| Palm Kernel | 95 |
| Babassu | 95 |
| Cocoa Butter | 201 |
| Cupu Assu | 95 |
| Sheanut | 357 |
| Cottonseed | 324 |
| Teaseed | 102 |
| Tomatoseed | 100 |
| Rice Bran | 1190 |
| Wheat Germ | 553 |
| Peanut | 207 |
| Soybean | 250 |
| Sesame | 865 |
| Olive | 221 |
| Poppyseed | 276 |
| Corn | 968 |
| Sunflower | 100 |
| Grapeseed | 180 |
| Walnut | 176 |
| Almond | 266 |
| Hazelnut | 120 |
| Apricot Kernel | 266 |
| Safflower | 444 |

In at least one embodiment, the source of the at least one phytosterol of the present disclosure is from vegetable oil.

Plant sterols may also be hydrogenated to produce plant stanols. Accordingly, the plant stanols of the present disclosure may be described as the hydrogenation products of the various plant sterols such as sitosterol, but may also be obtained naturally from various plants used in the art, without hydrogenating the plant sterol. Thus, the term “hydrogenation product of plant sterols” as applied to plant stanols, and as used herein, includes not only the synthetic plant stanols but also those obtained from natural sources. For example, mention may be made of plant stanols including sitostanol, campestanol, stigmastanol, spinostanol, taraxastanol, brassicastanol, desmostanol, chalinostanol, poriferastanol, clionastanol, and ergostanol. The skilled artisan may also select any plant stanol from those that are available. The disclosure may also employ mixtures of plant stanols, such as two component, three component, and four component mixtures, as well as mixtures of plant sterols and plant stanols such as two component, three component, and four component mixtures.

Both the plant sterols and plant stanols include the various position isomers and stereo isomeric forms used in the art, such as the α and βisomers as well as plant sterols and plant stanols that contain small (from one to about four carbon atom) side chains. For example, isomers β-sitosterol and β-sitostanol, respectively, may each be used as the at least one phytosterol. In one embodiment, the at least one phytosterol is a mixture of free plant sterols comprising β-sitosterol, compesterol, and stigmasterol from vegetable oil.

Administering phytosterols is attractive because phytosterols are naturally occurring compounds and the body essentially does not absorb them, which results in their elimination through normal excretion. Thus, preventing or reducing c-reactive protein levels, e.g., associated with vascular inflammation, through dietary routes with at least one phytosterol is desirable.

In at least one embodiment, the composition of the present disclosure is a beverage. The present disclosure, however, is not limited solely to the administration of a beverage; rather, it is contemplated as provided above that the composition according to the present disclosure may be in other forms, such as a pharmaceutical, a nutraceutical, and/or a solid or semi-solid consumable food product.

As such, when the composition is a beverage, it comprises a c-reactive protein level reducing amount of at least one phytosterol chosen from plant sterols and plant stanols. A process for producing a substantially stable dispersion to be used in a beverage comprises at least one phytosterol and an aqueous material, such as an aqueous beverage concentrate, such as a juice concentrate, as described, for example, in U.S. Patent Application Publication Nos. 2003/0232118 and 2004/0142087, the contents of which are incorporated herein by reference. The process comprises mixing the at least one phytosterol with the aqueous material to form a first dispersion. The next steps involve heating the first dispersion to form a heated mixture, followed by homogenizing the heated mixture to obtain a second dispersion of particles wherein the particle size of the at least one phytosterol in the first dispersion and the second dispersion is from about 0.1 microns to about 30 microns.

For example, the at least one phytosterol is incorporated into the beverage by mixing the at least one phytosterol with an aqueous beverage concentrate to form a first dispersion of particles that may be conducted at temperatures from about −10° C. to about 100° C. (about 14° F. to about 212° F.), or from about 0° C. to about 82° C. (about 32° F. to about 180° F.), or about 18° C. to about 64° C. (about 64° F. to about 148° F.), or about 24° C. to about 57° C. (about 75° F. to about 135° F.) for a period of time of from about 0.1 minutes to about 120 minutes, or from about 5 minutes to about 60 minutes, or from about 15 minutes to about 30 minutes, to form a first dispersion.

The apparatus employed for making the first dispersion of particles of the least one phytosterol and aqueous material, such as a beverage concentrate, comprises a high shear mixer (such as Arde-Barinco Model #CJ-4) or any large capacity (e.g., about 50 to about 300 gal.) high shear mixer. A commercial device for making the first dispersion may be, for example, a “Liquiverter” (Trademark) manufactured under the trade name APV Liquiverter model 200 CLV, manufactured by APV, an Invensys Company.

In at least one embodiment, the at least one phytosterol provided may be micronized to a size of about 0.5 microns to about 10 microns.

The particle size of the at least one phytosterol of both the first dispersion and the second dispersion may substantially follow a bell curve particle size distribution well known to a person with ordinary skill in the art.

The aqueous material can comprise water, water with additional compounds, and compositions dissolved or dispersed in it, either as a dispersion of solids in water or an emulsion of a liquid in water or water in a liquid. This defines the aqueous material of the disclosure, prior to mixing it with the at least one hydrophobic plant sterol. When employing the aqueous material with a dissolved or dispersed compound or composition, the solids content of the aqueous material, such as an aqueous beverage concentrate is from about 200 grams per liter of the aqueous material to about 1000 grams per liter of the aqueous material, or about 400 grams per liter to about 900 grams per liter, or about 600 grams per liter to about 800 grams per liter. “Solids content,” as that term applies to the “aqueous material” of the present disclosure, also may include any liquid added to the water used in forming an emulsion type of “aqueous material” as defined herein.

Mention may be made of other techniques to incorporate at least one phytosterol in a composition, for example:

Tiainen et al., U.S. Pat. No. 6,129,944, the contents of which are incorporated herein by reference, describes a method for producing a product containing a plant sterol by forming a homogeneous suspension of a microcrystalline plant sterol and a sweetening agent in an aqueous solution.

Haarasilta et al., WO 98/58554, the contents of which are incorporated herein by reference, describes a premix used in the food industry containing a pulverized plant sterol and a conventional foodstuff ingredient such as fruit, vegetable or berry type of material, particularly in a powder form and methods for preparing the premix. Grinding the plant sterol and the foodstuff such as berries, fruits, or vegetables according to methods and devices disclosed in Finnish patent applications FI 963 904 and FI 932 853, the contents of which are incorporated herein by reference, and with a grinder operating on the so-called impact milling principle, such as an Atrex mill manufactured by Megatrex Oy, produce this result. The inventors note that when applying the process of the invention to cereal in combination with a plant sterol, the temperature of the cereal grains rises due to the effect of mechanical energy on the grains, thereby providing heat treatment of the grains in conjunction with grinding.

Zawistowski, WO 00/45648, the contents of which are incorporated herein by reference, describes a method of preparing microparticles of plant sterols and plant stanols or mixtures of both by dispersing and suspending the plant sterols and plant stanols in a semi-fluid, fluid or viscous vehicle and exposing the vehicle so formed to impact forces. The method involves dispersing or otherwise suspending the plant sterol and/or plant stanol in a suitable semi-fluid, fluid or viscous vehicle followed by applying impact forces to the vehicle to produce microparticles. Zawistowski develops these impact forces by creating high-shear either with an air-atomization nozzle, a pneumatic nozzle, a high-shear mixer, or colloid mill, but preferably a microfluidizer commercially avail able from Microfluidics Incorporation, Newton, Mass.

According to the present disclosure, an effective amount of the at least one phytosterol for reducing CRP level is administered. As used herein, the term “c-reactive protein level reducing amount” means the at least one phytosterol concentration that has the ability to elicit a biological or medical response of a tissue, system, or subject that is being sought by the administrator, which may include the modulation, i.e., slowing or halting the progression of vascular inflammation and/or reduction of c-reactive protein levels.

To achieve such a composition with the at least one phytosterol, for example, the at least one phytosterol may be present in the first dispersion and/or the second dispersion in an amount from about 1 gram to about 100 grams per liter or from about 10 grams to about 60 grams per liter, or about 20 grams to about 30 grams per liter of the aqueous material, concentrate, or beverage product. In one embodiment, the at least one phytosterol may be present in the first dispersion and/or the second dispersion in an amount from about 15 grams to about 30 grams per liter of the aqueous material, concentrate, or beverage product.

A total daily dose of the at least one phytosterol, as well as the dose frequency, will vary depending on the particular dosage form used and the route of administration. The amount and frequency of administration will also vary according to age, body weight, and condition and response of the individual subject. Dosing and dosing frequency can be readily determined by a competent physician without undue experimentation. It is also noted that the clinician or treating physician will know how and when to interrupt, adjust, or terminate therapy in conjunction with individual subject response.

In general, the total daily dose is up to 2 gram or higher, or from about 1 mg to about 3 g, or from about 1 mg to 5 g, or from 1 g to 10 g or any amount in between these ranges. In an embodiment, the total daily dose of the at least one phytosterol may be up to 2 grams.

In an embodiment, when the at least one phytosterol is administered as a beverage, the at least one phytosterol may be present in an amount up to about 100%, such as from about 0.5% to about 80% and further for example, from about 1% to about 50% or any fraction in between these ranges, by weight relative to the total composition. As further example, when the at least one phytosterol is administered as a composition, the at least one phytosterol may be present in an amount up to 100%, such as from about 0.1% to about 75% or any fraction in between these ranges, by weight relative to the total weight of the composition.

The homogenizing of the first dispersion to obtain a second dispersion of particles of the at least one hydrophobic plant sterol and the aqueous beverage concentrate may be, for example, conducted in a homogenizer (such as, APV model # APV 1000), which may function by forcing the dispersion through a small orifice at high pressures. The homogenizing may be carried out at a pressure from about 100 psi to about 14,500 psi, or about 500 psi to about 10,000 psi, or about 1,000 psi to about 5,000 psi. In one embodiment, the homogenizing is carried out at a pressure of about 2,000 psi to about 5,000 psi.

Various beverage concentrates may be employed as the aqueous material, however, in one embodiment, the process involves producing a substantially stable dispersion comprising at least one phytosterol and an aqueous citrus juice concentrate such as an orange juice concentrate.

In one embodiment, the aqueous material comprises water, and water in combination with nutrients, flavorants, sweeteners, carbon dioxide and other gases, and combinations thereof. Further for example, the aqueous material may be, but is not limited to, a concentrate of a fruit juice, or fruit flavor, such as citrus juices including orange, lemon, lime, tangerine, mandarin and, grapefruit juice, and other juice and fruit flavor concentrates such as acerola, grape, pear, passion fruit, pineapple, banana, apple, cranberry, cherry, raspberry, peach, plum, grape, currant, cranberry, blackberry, blueberry, strawberry, mirabelle, watermelon, honeydew, cantaloupe, mango, papaya, botanical flavors such as flavors derived from cola, tea, coffee, chocolate, vanilla, almond, vegetable juices and flavors such as tomato, cabbage, celery, cucumber, spinach, carrot, lettuce, watercress, dandelion, rhubarb, beet, cocona, guava, han guo, and mixtures thereof, such as two component, three component and four component mixtures.

The aqueous material of the present disclosure may also comprise concentrates of typical sport beverages, and beverages used to treat loss of fluids due to illness, and which contain sucrose syrup, glucose-fructose syrup, citric acid, sodium citrate, mono-potassium phosphate and potassium salts, and other materials for replenishing lost electrolytes, whether as a product requiring the addition of water or in admixture with water.

The concentrates of the present disclosure may be diluted with water to form juices or drinks. For example, where the concentrate includes a sugar or mixture of sugars, it can be diluted with water to about 2° Brix to about 20° Brix, or about 6° Brix to about 16° Brix, or about 11° Brix to about 13° Brix. The sugars employed according to the present disclosure may generally comprise carbohydrate materials such as fructose, sucrose, glucose and the like as well as the other sugars used in the art as described by McMurry, Organic Chemistry, Third Edition, pp. 916-950, Hawley's Condensed Chemical Dictionary, Twelfth Edition, p. 1100, and Hackh's Chemical Dictionary, Third Edition, pp. 815-817. In addition, non-nutritive high intensity sweeteners, natural or artificial sweeteners can also be employed. Mixtures of sugars and/or sweeteners can also be used, such as two component, three component, or four component mixtures.

The compositions contemplated by the present disclosure may contain a variety of optional components. Such optional components may be dispersed, solubilized, or otherwise mixed into the various forms of the composition, i.e., a pharmaceutical composition or other consumable product. Non-limiting examples of optional components suitable for use herein are provided below.

When the composition is a pharmaceutical composition, optional components may include, but are not limited to, carriers, fillers, extenders, binders, disintegrating agents, solution-retarding agents, absorption accelerators, wetting agents, absorbents, lubricants, stabilizers, coloring agents, buffering agents, dispensing agents, preservatives, organic acids, water-soluble and water-insoluble polymers, enteric agents and non-enteric agents, coatings, and any other ingredient or ingredients typically used as optional pharmaceutical components.

When the composition is a consumable product, such as a food product or a beverage, optional components may include, but are not limited to, nutrients such as vitamins and minerals, flavorants, coloring agents, carbonation components, preservatives, gums, emulsifiers, and any other ingredient or ingredients typically used as optional consumable product components.

For example, the composition of the present disclosure may comprise at least one water soluble vitamin, such as vitamin C, vitamin B6 and/or vitamin B12, folic acid, and/or at least one oil soluble vitamin such as vitamin A, beta carotene, vitamin B, e.g., the D vitamins, vitamin E, and vitamin K, such as two component, three component, and four component mixtures. The addition of a vitamin, such as vitamins B and E varies to obtain an RDA from about 1% to about 100%, or about 5 to about 30%, or about 15 to about 20% of the RDA for each vitamin per unit serving.

C-reactive protein assays and methodologies are known to those skilled in the relevant art. In addition, methods for analyzing c-reactive protein levels are described in U.S. Pat. Nos. 5,358,852, 6,040,147, and 6,277,584, the contents of which are incorporated herein by reference. Highly sensitive assays for CRP are commercially available from several vendors such as Dade Behring, Inc., Abbot Laboratories, and Roche Laboratories.

For example, the levels of CRP can be measured by using a high sensitivity CRP (hs-CRP) assay performed using a Beckman LX20PRO with a highly sensitive Near Infrared Particle Immunoassay Rate (NIPIA) methodology. According to this method, an anti-CRP antibody-coated particle binds to CRP in the plasma sample resulting in the formation of insoluble aggregates, which cause turbidity. See Product Brochure for High Sensitivity C-Reactive Protein (CRPH) by Beckman Coulter. By monitoring the change in absorption at 940 nm, one can determine the concentration of CRP in a sample, i.e., the change in absorbance is proportional to the concentration of CRP. The LX PRO system expresses CRP concentration based upon a single-point adjusted, pre-determined calibration curve.

The present disclosure further contemplates the addition of at least one active agent other than the at least one phytosterol to the composition, such as compounds that may be able to treat the same condition being treated with the at least one phytosterol, e.g., the addition of at least one statin, as well as different, or related conditions. Such active agents include, but are not limited to, 3-hydroxy-3-methylglutaryl coenzyme A reductase inhibitors (statins), peroxisome proliferators-activated receptor-α agonists (fibrates), peroxisomes proliferators-activated receptor-α agonists (glitazones), aspirin, and high doses of RRR-α tocopherol. The present disclosure also contemplates the at least one phytosterol administered as a mono-therapy, i.e., the administration of the at least one phytosterol alone.

Alternatively, when such additional agents may be provided, they may be in a separate formulation and co-administered to a subject with the composition of the present disclosure. Such separate formulations may be administered before, after, or simultaneously with the administration of the composition of the present disclosure.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The examples below serve to illustrate the present disclosure in a non-limiting manner.

Preparation of Phytosterol Beverage

Combining the following components provided a base mixture of phytosterols with an aqueous material before subsequent processing to form a first dispersion of a beverage of the present disclosure.

The composition was formulated to obtain the following:

Base Ingredients:

| | |
|---|---|
| Desired Volume | 0.75 gallons |
| Water | 180.3 grams |
| Orange concentrate | 3363.0 grams |

(Refractometer °Brix, 65 (corrected for acid); acid 3.71% (wt./wt.))

| | |
|---|---|
| Orange flavor | 53.1 grams |
| Orange oil | 2.7 grams |
| Phytosterol | 76.7 grams |
| Total | 3675.8 grams |

Finished Product Ingredients:

| | |
|---|---|
| Desired Volume | 4.8 gallons |
| Water | 4.05 gallons |
| Base | 0.75 gallons |

| Base Specifications | Optimum | Minimum | Maximum |
|---|---|---|---|
| Percent Soluble Solids | 61.56 | 61.14 | 62.39 |
| Refractometer Brix | 61.15 | 60.74 | 61.99 |
| % Acid w/w as citric acid | 3.42 | 3.12 | 3.72 |
| Brix/Acid Ratio | 18.0 | 16.4 | 20.0 |

The substantially stable dispersion of the at least one phytosterol and the orange juice concentration as the aqueous material had a concentration of 61.15 Brix (refractometer Brix, corrected for acid).

The mixture was stirred using an Arde-Barinco Model No. CJ-4 high shear mixer at 7000 rpm for about 15 minutes and heated to 82.2° C. (180° F.) in eight seconds and chilled to about 43.3° C. to about 60° C. (about 110° F. to 140° F.) in about five seconds to produce a first dispersions having an average particle size of about 10 microns and a particle size distribution of about 0.5 microns to about 30 microns with the maximum particle size being about 30 microns.

Homogenizing the first dispersion in an APV homogenizer, Model No. APV 1000 from the APV Homogenizer Group (an Invensys Co.) at 60° C. (140° F.) at 3400 psi and then 600 psi produced the second dispersion.

The second dispersion comprised a substantially stable dispersion comprising the at least one phytosterol and the orange juice concentrate as the aqueous material. Adding water to the substantially stable dispersion produced an orange juice product of 12.00° Brix. The product is manufactured to the following specifications:

| Product Specifications | Optimum | Minimum | Maximum |
|---|---|---|---|
| Percent Soluble Solids | 12.00 | 11.90 | 12.20 |
| Refractometer Brix | 11.92 | 11.82 | 12.12 |
| % Acid w/w as citric acid | 0.67 | 0.65 | 0.69 |
| Brix/Acid Ratio | 18.0 | 17.3 | 18.8 |

Study I: Phytosterol Beverage

Seventy-two subjects participated in this placebo-controlled, double-blinded, randomized trial. Sridevi Devaraj et al., Plant Sterol-Fortified Orange Juice Effectively Lowers Cholesterol Levels in Mildly Hypercholesterolemic Healthy Individuals, 24 Artherioscler. Thromb. Vasc. Biol. e24-e28 (2004), the contents of which are incorporated herein by reference. Adults with normal complete blood counts, LDL-cholesterol >100 mg/dL; normal liver and renal function (normal transaminases, alkaline phosphatase, creatinine); no bleeding diathesis, normal thyroid function (normal TSH) were included in the study.

All subjects underwent a two-week run-in phase in which they received an unfortified beverage. They were then randomized in a blinded fashion to receive phytosterol fortified beverage (i.e., phytosterol beverage group) or placebo beverage for the next eight weeks. The phytosterol beverage with a targeted particle size distribution was suspended in a juice using the process as exemplified above. Subjects were given enough beverage to last 18 days. Analytical studies demonstrated that the finished beverage showed phytosterol remained in the beverage throughout shelf-life.

Each subject was asked to consume 240 mL of the beverage twice daily with meals. This corresponds to approximately 2 g per day of phytosterol in the phytosterol beverage.

In addition, subjects were asked to refrain from consuming any other source of fortified margarines such as Benecal® or Take Control®, 4 weeks prior to study entry and during the period of the study. Fasting blood samples were obtained at baseline (average of 2 samples, 5 to 7 days apart), after two weeks, and after 10 weeks of the study (average of 2 samples, 5 to 7 days apart). The composition of the placebo beverage and phytosterol beverage are provided in Table II below.

TABLE II
Composition of the Phytosterol and Placebo Beverage

| Ingredient | Phytosterol Beverage | Placebo Beverage |
|---|---|---|
| Calories | 110 | 110 |
| Folate (μg) | 60 | 60 |
| Vitamin E (IU) | 6 | 0 |
| Vitamin B6 (mg) | 0.4 | 0.08 |
| Vitamin B12 (μg) | 1.2 | 0 |
| Vitamin C (mg) | 72 | 72 |
| Potassium (mg) | 450 | 450 |
| Thiamin (mg) | 0.15 | 0.15 |
| Free Sterol | 1.0 | 0 |

To examine the CRP levels of the respective samples taken during the course of the study, plasma was separated from red blood cells after 15 minutes of centrifugation at 600 g. All analyses were carried out using the Near Infrared Particle Immunoassay Rate methodology on a Beckman LX20PRO system. While 75 subjects entered the study, 3 dropped out due to personal reasons (two in the phytosterol group and one in the placebo group) and 72 subjects (n=36/group) completed the study. Subjects in both groups were matched for age, gender, and body mass index. In Table III, the CRP values are provided for the subjects in both groups.

TABLE III
Summary of the CRP levels for the
Placebo and Phytosterol Beverage

| | | CRP value (mg/L) | | | | |
|---|---|---|---|---|---|---|
| | | Min- | Max- | | | |
| | | imum | imum | Mean | Median | P-value* |
| Placebo | Baseline | 0.2 | 10.80 | 2.771 | 1.70 | 0.7550 |
| (n = 35) | Post- | 0.2 | 12.30 | 2.754 | 1.70 | |
| | Adminis- | | | | | |
| | tration | | | | | |
| Phytosterol | Baseline | 0.2 | 9.80 | 2.597 | 1.50 | <0.0001 |
| Beverage | Post- | 0.2 | 7.20 | 1.808 | 1.15 | |
| (n = 36) | Adminis- | | | | | |
| | tration | | | | | |

*Based on the Wilcoxon's signed rank test.

The Wilcoxon's signed rank test was used for statistical comparisons with the placebo and phytosterol beverage at baseline and post-administration to evaluate changes in CRP values. See Wilcoxon, F. Individual Comparisons by Ranking Methods, 1 Biometrics 80-83 (1945). The Wilcoxon's signed rank test is often used to test differences of data collected before and after an investigation and is an alternative to the paired Student's t-test. The analysis of the phytosterol beverage at baseline compared to post-administration resulted in a P value of <0.0001. The P value is an estimated probability of rejecting the null hypothesis (i.e., there would be no difference between CRP levels at baseline and post-administration) when the hypothesis is true. Meaning, it attempts to measure the strength of the results of the test.

Generally, P values of <0.05 indicate statistical significance and P values <0.001, i.e., less than one thousand chance of being wrong, indicates statistically high significance. In this case, given the small P value, the null hypothesis may be false. Considering that the study was double-blinded, the P value of <0.0001 suggests that the results are unlikely due to chance and are of high statistical significance, i.e., the reduction in CRP is not due to chance.

Study II: Reduced Calorie Phytosterol Beverage

Seventy-two subjects participated in this placebo-controlled, double-blind randomized trial. Adults with normal complete blood count (CBC), LDL-cholesterol >100 mg/dL; normal liver and renal function (normal transaminases, alkaline phosphatase, creatinine); no bleeding diathesis, normal thyroid function (normal TSH) were included in the study.

Subjects were randomized in a blinded fashion to receive a reduced calorie beverage with phytosterols comprising at least 2%, by weight relative to the total beverage composition or a placebo for the next 8 weeks. Phytosterol with targeted particle size distribution was suspended in reduced-calorie beverage, as described above. Subjects were given enough beverage to last 18 days.

Each subject was asked to consume 240 ml of beverage, twice daily with meals. This corresponded to approximately 2 grams per day of phytosterol. In addition, subjects were asked to refrain from consuming any other source of fortified margarines such as Benecol® Take Control®, 4 weeks prior to study entry and during the period of the study.

Fasting blood samples were obtained at baseline (average of 2 samples, 5-7 days apart), after 4 weeks, after 8 weeks of the study (average of 2 samples, 5-7 days apart). The composition of the placebo and phytosterol beverage are given in Table IV.

TABLE IV
Composition of Beverages

| Ingredient | Phytosterol Beverage | Placebo Beverage |
|---|---|---|
| Calories | 50 | 50 |
| Total Fat (g) | 0 | 0 |
| Total Carbohydrate (g) | 13 | 13 |
| Total Protein (g) | 0 | 0 |
| Folate (μg) | 24 | 24 |
| Vitamin E (IU) | 6 | 0 |
| Vitamin B6 (mg) | 0.4 | 0.08 |
| Vitamin B12 (μg) | 1.2 | 0 |
| Vitamin C (mg) | 72 | 72 |
| Potassium (mg) | 450 | 450 |
| Thiamin (mg) | 0.15 | 0.15 |
| Free Sterol | 1.0 | 0 |

To examine the CRP levels of the respective samples taken during the course of the study, plasma was separated from red blood cells after 15 minutes of centrifugation at 600 g. All analyses were carried out using the Near Infrared Particle Immunoassay Rate methodology on a Beckman LX20PRO system. While 77 subjects entered the study, 5 dropped out due to personal reasons (two in the phytosterol group and three in the placebo group) and 72 subjects (n=36/group) completed the study. Subjects in both groups were matched for age, gender, and body mass index. In Table V, the CRP values are provided for both groups.

TABLE V
Summary of CRP levels in the Placebo and Reduced
Calorie Phytosterol Beverage groups

| | | CRP value (mg/L) | | | | |
|---|---|---|---|---|---|---|
| | | Min- | Max- | | | |
| | | imum | imum | Mean | Median | P-value* |
| Placebo | Baseline | 0.200 | 7.50 | 2.723 | 1.800 | 0.3821 |
| (n = 35) | Post- | 0.200 | 10.70 | 3.289 | 1.900 | |
| | Adminis- | | | | | |
| | tration | | | | | |
| Phytosterol | Baseline | 0.300 | 10.40 | 2.803 | 1.700 | 0.0006 |
| Beverage | Post- | 0.200 | 8.20 | 1.966 | 1.500 | |
| (n = 35) | Adminis- | | | | | |
| | tration | | | | | |

*Based on the Wilcoxon's signed rank test.

As provided from the comparison between the placebo and phytosterol beverage at baseline and post-administration, the median CRP level was reduced in the reduced calorie phytosterol beverage, as compared to the placebo. Using the Wilcoxon's signed rank test, the analysis of the reduced calorie phytosterol beverage at baseline compared to post-administration resulted in a P value of 0.0006. Thus, given the small P value and that the study was double-blinded, the null hypothesis may be rejected and the reduction in CRP may not be likely due to chance.

What is claimed is:

1. A method for reducing the level of c-reactive protein comprising administering to a subject in need thereof a c-reactive protein level reducing amount of at least one phytosterol.

2. The method according to claim 1, wherein the at least one phytosterol is administered orally.

3. The method according to claim 1, wherein the at least one phytosterol is administered as a component of a composition.

4. The method according to claim 3, wherein the composition is chosen from a pharmaceutical composition and a consumable product.

5. The method according to claim 4, wherein the consumable product is chosen from a food and a beverage.

6. The method according to claim 5, wherein the consumable product is a beverage.

7. The method according to claim 6, wherein the beverage is a citrus juice.

8. The method according to claim 1, wherein the at least one phytosterol is administered to achieve a total daily dose of about 2 grams per day.

9. The method according to claim 8, wherein the dose is chosen from a single or divided dose.

10. The method according to claim 1, further comprising repeating the administration of the at least one phytosterol.

11. The method according to claim 1, wherein the at least one phytosterol is chosen from plant sterols and plant stanols.

12. The method according to claim 1, wherein the at least one phytosterol is chosen from sitosterol, campesterol, spinosterol, taraxasterol, brassicasterol, demosterol, chalinosterol, poriferasterol, clinosterol, ergosterol, and mixtures thereof.

13. The method according to claim 1, wherein the at least one phytosterol is chosen from free plant sterols and free plant stanols.

14. The method according to claim 1, wherein the at least one phytosterol is derived from vegetable oil sterols.

15. The method according to claim 1, wherein the at least one phytosterol is a mixture of free plant sterols comprising beta-sitosterol, campesterol, and stigmasterol.

16. The method according to claim 1, wherein the subject is a human.

17. The method according to claim 1, further comprising reducing serum cholesterol levels with the administration of the c-reactive protein level reducing amount of at least one phytosterol.

18. A method for reducing the level of c-reactive protein comprising administering to a subject in need thereof a composition comprising a c-reactive protein reducing amount of at least one phytosterol.

19. The method according to claim 18, wherein the composition is chosen from a pharmaceutical composition and a consumable product.

20. The method according to claim 18, wherein the composition further comprises an optional component chosen from carriers, fillers, extenders, binders, disintegrating agents, solution-retarding agents, absorption accelerators, wetting agents, absorbents, lubricants, stabilizers, coloring agents, buffering agents, dispensing agents, preservatives, organic acids, water-soluble and water-insoluble polymers, enteric agents and non-enteric agents, coatings, nutrients, flavorants, coloring agents, carbonation components, preservatives, gums, emulsifiers, and mixtures thereof.

21. The method according to claim 18, wherein the composition further comprises at least one active agent other than the at least one phytosterol.

22. A method for treating or preventing vascular inflammation comprising administering to a subject in need thereof a c-reactive protein level reducing amount of at least one phytosterol.

23. A method for reducing the level of c-reactive protein comprising administering to a subject in need thereof a beverage comprising a c-reactive protein level reducing amount of at least one phytosterol.

24. A method for reducing the level of c-reactive protein comprising administering to a subject in need thereof a mono-therapy comprising a c-reactive protein level reducing amount of at least one phytosterol.

25. A method for reducing the level of c-reactive protein comprising administering to a subject in need thereof a beverage comprising a substantially stable dispersion of at least one phytosterol in a c-reactive protein level reducing amount and an aqueous material wherein the at least one phytosterol is chosen from plant sterols and plant stanols, wherein in order to avoid a powdery taste in the substantially stable dispersion, the particle size of the at least one phytosterol is from 0.1 micron to about 30 microns and a majority of the at least one phytosterol particles are within a range from about 0.2 microns to about 10 microns and follow a bell curve distribution.
